(19) United States
(12) Patent Application Publication
(10) Pub. No.: US 2014/0121485 A2
(43) Pub. Date: May 1, 2014

(54) Electrochemical Transdermal Glucose Measurement System Including Microheaters and Process For Forming

(71) Applicants: Makarand Paranjape, Silver Spring, MD (US); Arend Jasper Nijdam, Lorton (US); Yogesh Ekanath Kashte, Mumbai, Maharashtra (IN)

(72) Inventors: Makarand Paranjape, Silver Spring, MD (US); Arend Jasper Nijdam, Lorton, VA (US); Yogesh Ekanath Kashte, Maharashtra (IN)

(73) Assignee: Georgetown University, Washington, DC (US)

(21) Appl. No.: 13/459,392

(22) Filed: Apr. 30, 2012

(51) Int. Cl. A61B 5/1477; C25D 5/02

(52) U.S. Cl. 600/347; 600/345; 205/136

(57) ABSTRACT

A device contains individually controllable sites for electrochemically monitoring an analyte in interstitial fluid of a user. The sites include a conductive pattern attached at a first and second ends thereof to electrode material in a closed-circuit configuration for receiving a first predetermined voltage applied thereto in order to thermally ablate a stratum corneum of a user's skin to access the interstitial fluid and form an open-circuit configuration including first and second portions of the electrode material that are electrically isolated from each other; a sensing area deposited on at least one of the first and second portions of the electrode material; and a measuring component for receiving individual measurement data from the sensing area in response to a second predetermined voltage applied to the open circuit configuration. The individual measurement data is indicative of an amount of the analyte in the interstitial fluid.

### BACKGROUND

1. Field of Embodiments

The present embodiments relate generally to non-invasive or minimally invasive transdermal measurement systems. More specifically, the embodiments relate to non-invasive or minimally invasive transdermal glucose measurement systems and processes for forming.

2. Summary of Existing Art

Minimally invasive transdermal systems are described in, for example, co-owned U.S. Pat. Nos. 6,887,202 and 7,931,592 both entitled “Systems and Methods for Monitoring Health and Delivering Drugs Transdermally,” which are incorporated herein by reference in their entirety.

These systems, like the embodiments described herein, provide for a minimally invasive sampling technique and device suitable for rapid, inexpensive, unobtrusive, and pain-free monitoring of important biomedical markers, such as glucose. Existing systems remain open to improvement, particularly with respect to size or footprint, as the systems may be intended to be worn by a person under their clothing. Obviously this application would benefit from a device having a small footprint so as to remain inconspicuous. Similarly, the ability to fit multiple sampling sites on a single device is also desired, facilitating continuous and timely monitoring and reducing the need for user to take affirmative action until the all sampling sites on the device are exhausted.

### BRIEF SUMMARY OF EMBODIMENTS

In a first embodiment, a device containing at least two individually controllable sites for electrochemically monitoring an analyte in interstitial fluid of a user includes: a glass substrate having formed thereon at each of the at least two individually controllable sites:

a serpentine conductive pattern attached at a first and second ends thereof to electrode material in a closed-circuit configuration for receiving a first predetermined voltage applied thereto in order to; i. thermally ablate a stratum corneum of a user's skin to access, the interstitial fluid of the user and ii. form an open-circuit configuration including first and second portions of the electrode material that are electrically isolated from each other; a sensing area deposited on at least one of the first and second portions of the electrode material; and a measuring component for receiving individual measurement data from the sensing area in response to a second predetermined voltage applied to the open circuit configuration of each of the at least two individually controlled sites in the open-circuit configuration, wherein the individual measurement data is indicative of an amount of the analyte in the interstitial fluid of the user.

In a second embodiment, a process for electrochemically monitoring an analyte in interstitial fluid of a user includes: applying a first predetermined voltage to a closed-circuit device located proximate to a portion of skin of the user that includes a serpentine conductive pattern attached at a first and second ends thereof to electrode material in order to i. thermally ablate a stratum corneum of, a user's skin to access the interstitial fluid of the user; and ii. separate the electrode material to form an open-circuit device including first and second portions of the electrode material that are electrically isolated from each other; applying a second predetermined voltage to the open-circuit device which is electrically contacted with the interstitial fluid; and receiving at a measuring component from a sensing area located on at least one of the first and second portions of the electrode material, measurement data indicative, of an amount of the analyte in the interstitial fluid of the user.

In a third embodiment, a device contains at least two individually controllable sites for electrochemically monitoring an analyte in interstitial fluid of a user including: a glass substrate having formed thereon at each of the at least two individually controllable sites: a serpentine conductive pattern attached at a first and second ends thereof to electrode material in a closed-circuit configuration for receiving a predetermined voltage applied thereto in order to thermally ablate a stratum corneum of a user's skin to access the interstitial fluid of the user; a sensing area located on at least a portion of the electrode material; and first and second measuring electrodes for obtaining measurement data from the sensing area; and a measuring component for receiving individual measurement data from the first and second measuring electrodes of each of the at least two individually controlled sites, wherein the individual measurement data is indicative of an amount of the analyte in the interstitial fluid of, the user.

In a fourth embodiment, a process for electrochemically monitoring an analyte in interstitial fluid of a user includes: applying a first predetermined voltage to a closed-circuit device located proximate to a portion of skin of the user that includes a serpentine conductive pattern attached at a first and second ends thereof to electrode material in order to thermally ablate a stratum corneum of a user's skin to access the interstitial fluid of the user and form an open-circuit device; applying a second predetermined voltage to the open-circuit device which is in electrical contact with the interstitial fluid; measuring an electrochemical response resulting from an interaction of the analyte with a sensing layer on a portion of the electrode material; and receiving at a measuring component from the open circuit device, measurement data, indicative of an amount of the analyte in the interstitial fluid of the user.

In a fifth embodiment, a process for forming a device containing at least two individually controllable site for electrochemically monitoring glucose in interstitial fluid of a user includes: depositing a first layer of one of chrome or titanium on a glass substrate; depositing a second layer of one of gold or platinum on the first layer of chrome; patterning the first and second layers in a first predetermined pattern to form multiple electrodes; depositing polymethyl methacrylate (PMMA) on the first predetermined pattern; patterning the PMMA in a second predetermined pattern, wherein at least a portion of the first predetermined pattern is exposed; and electrochemically depositing glucose oxidase on the exposed portion of the first predetermined pattern.

### BRIEF DESCRIPTION OF FIGURES

The following figures are intended to exemplify the various embodiments described herein and are in no away intended to be limiting.

FIGS. 1(a) to 1(i) are representative of the various stages of manufacture of a device as described with respect to a first embodiment;

FIGS. 2(a) to 2(h) are representative of the various stages of manufacture of a device as described with respect to a second embodiment;

FIGS. 3(a)-3(b) are representative of normal masks used in accordance with the embodiments described herein;

FIGS. 3(c) is representative of a shadow mask used in accordance with the embodiments described herein;

FIG. 4 is representative of devices formed in accordance with the embodiments described herein;

FIG. 5 indicates the inflection point I used to determine an appropriate voltage for electrodeposition in accordance with at least one step of the embodiments described herein;

FIG. 6 is illustrative of polypyrole deposition at selected voltage (0.6 V) for 60 seconds in accordance with at least one step of the embodiments described herein;

FIG. 7 is illustrative of multiple CV scan runs from −1 V to +1 V to verify one or more depositions and establish polarization potential in accordance with at least one step of the embodiments described herein; and

FIGS. 8a through 8d illustrate various dimensions: of representative devices in accordance with a preferred embodiment herein.

### DETAILED DESCRIPTION

The processes described herein are used to form an array of individual monitoring sites. The array may be applied to a person's skin, e.g., in the form of an adhered patch, and each individual monitoring site may be controlled to collect interstitial fluid at different times. Such a monitoring system is useful for people who live with a condition, such as diabetes, wherein frequent glucose measurements are required in order to maintain health.

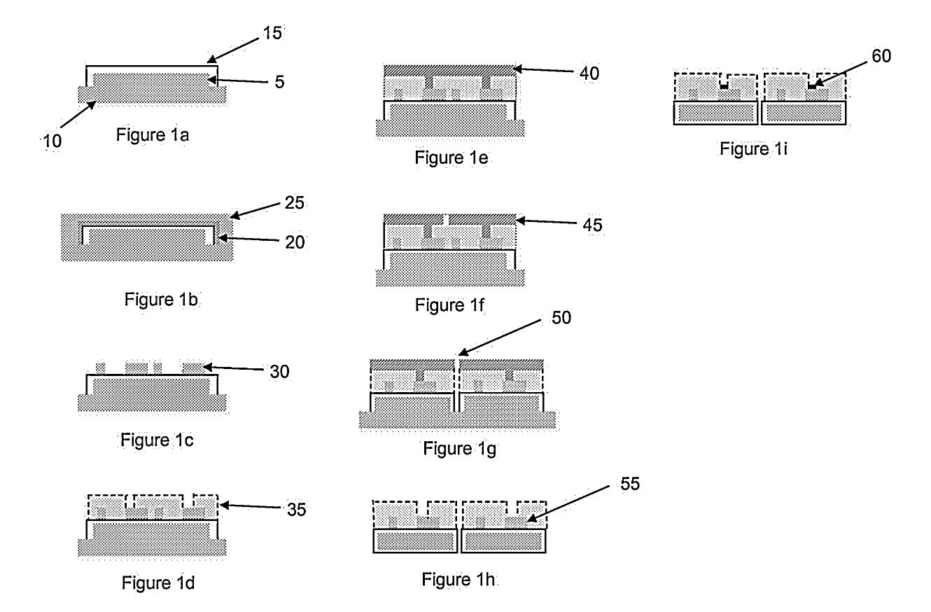

A first exemplary process for forming arrays of transdermal monitoring sites is described with reference to FIGS. 1(a)-1(i). Micro and nano-fabrication processes are utilized to form a macro device, e.g., on the order of a centimeters in total size, that is comprised of numerous micro and nano-sized layers and components. In this first exemplary embodiment, the major fabrication steps generally include: Clean, back and mark wafers; deposit chrome and gold; pattern chrome and gold through standard lithography and wet etching; deposit PMMA and pattern PMMA through a shadow mask with an oxygen plasma; deposit aluminum; pattern aluminum standard lithography and wet etching; plasma etch deep trenches; remove aluminum; deposit glucose oxidase electrochemically; pattern PMMA through a shadow mask with an oxygen plasma. These steps are described more specifically below and with reference to FIGS. 1(a)-1(i) (figures are not to scale).

Initially, as shown in FIG. 1(a), a selected primary wafer formed of silicon is cleaned and marked. For example, a piraña clean which is H₂SO₄:H₂O₂=4:1 and applied for 20 minutes @80° C. may be used to remove organic contaminants from the primary wafer 5. The thickness of the wafers may require that they be adhered to a carrier wafer 10 for structural stability during the fabrication process. In this embodiment, the approximately 150 μm primary wafers are glued to carrier wafer of comparable material using a photoresist (PR) as glue, e.g., 4 mL Shipley 1813 PR and baked for approximately 45 minutes at 50° C. The primary wafers have an approximately 1 μm silicon oxide layer on the front side 15. The wafers are marked using known techniques for identification throughout the preparation process.

The next step as shown in FIG. 1(b) is a chrome/gold deposition. Chrome 20 is needed as an intermediate layer as gold 25 has poor adhesion to silicon oxide. The chrome and gold are sputtered using a standard plasma deposition machine. Layer details are set forth in Table 1 below. As an alternative to gold, platinum may be used

TABLE 1

200 Å Cr or Ti sputter deposition
5000 Å Au sputter deposition

Referring to FIG. 1(c), the chrome and gold are patterned 30 through standard lithography and wet etching in order to form the metal leads for the array. Table 2 sets forth recipe and layer formation details. In this embodiment, commercially available Shipley photoresist (PR) and Transene chrome (TFN) and gold (TFA) etch are used.

TABLE 2

Spin ~1 mL Hexamethyldisiloxane (HMDS)
Spin Shipley ~2 mL S1813 PR
Bake 60 s @ 110° C.
Expose 10 s
Develop 20-40 s in CD-30, (FRESH DEVELOPER)
Bake 30 min @ 120° C.
Gold etch: Use TFA etchant
Immerse 2-5 min until completely etched, (FRESH ETCHANT)
Rinse with De-Ionized water, dry
Chrome etch: mix Transene TFN etchant to DI water
Immerse 1-2 min until completely etched, (FRESH ETCHANT)
Rinse with De-Ionized water, dry

Referring to FIG. 1(d), Polymethyl methacrylate (PMMA) is deposited and patterned as a mask 35 for the future deposition of glucose oxidase. PMMA is spun and baked for the deposition. Layer thickness is monitored using the reflectometer. The PMMA layer is patterned in an oxygen plasma by use of a manually aligned steel shadow mask. Table 3 sets forth recipe and layer formation details.

TABLE 3

Spin ~2 mL 950 Shipley PMMA C2
Prebake 90 s @ 180° C.
Reactive Ion Etching O₂ etch, use shadow masks

Next, approximately 500 Å aluminum 40 is deposited in a sputter process as shown in FIG. 1(e). The aluminum will function as a mask defining the chip shape in a future plasma etching step. Alignment marks included in the chrome gold pattern are covered with tape to stay visible and allow alignment of the pattern in the next step.

In FIG. 1(f), the aluminum is patterned 45 using lithography and wet etching to shape the individual array patterns therein. The aluminum is etched using a solution of phosphoric acid, and a bit of nitric acid, acetic acid, and water as exemplified in Table 4 below.

TABLE 4

Spin ~1 mL HMDS
Spin Shipley ~2 mL Shipley 1813 PR
Bake 60 s @ 120° C. in oven
Expose 10 s
Develop 20-40 s in CD-30, (FRESH DEVELOPER)
Bake 30 min @ 120° C. in oven
Mix Al etch, (FRESH ETCHANT):
85 mL 85% H₃PO_{4,} 5 mL 70% HNO₃, 5 mL glacial
HAc, 5 mL DI water
Heat to 40-50° C.
Immerse for 45-60 sec until completely etched
Rinse with DI water, dry

Next, as shown in FIG. 1(g), plasma etching is used to etch deep trenches 50. First, short oxygen plasma is applied to etch the PMMA. Then, the silicon oxide layer is etched using an inductively coupled plasma (ICP) process, e.g., Bosch process. Finally, the silicon wafer is etched using the Bosch process which is known to those skilled in the art.

And in FIG. 1(h), all remaining aluminum is etched using the same wet etch described in Table 4 to expose first electrodes in the arrays 55. The recipe and steps are identified in Table 5 below.

TABLE 5

Mix Al etch, (FRESH ETCHANT):
85 mL 85% H₃PO_{4,} 5 mL 70% HNO₃, 5 mL glacial
HAc, 5 mL DI water
Heat to 40-50° C.
Immerse for 45-60 sec until completely etched
Rinse with DI water, dry

Finally, glucose oxidase is electrochemically deposited 60 through the openings in the PMMA layer as shown in FIG. 1(i). The recipe and steps are identified in Table 6 below.

TABLE 6

Prepare solution of 0.1M pyrole and 0.1M KCl
(or 0.1M NaDBS) in PBS
Immerse sample in solution, apply 0.6 V, constant current
Add 18 μL GOx and 48 μL in 10 mL PBS for
incorporation of GOx and redox mediator

In a final step (not illustrated), the second electrode is opened up in the PMMA layer using the same oxygen plasma specifications and mask as described in the last two steps of Table 3.

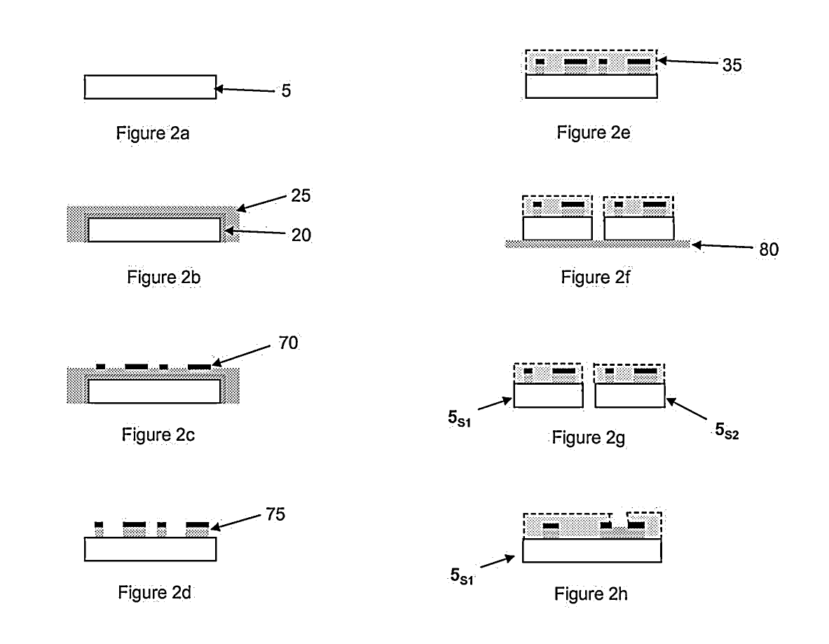

A second exemplary process for forming arrays of transdermal monitoring sites is described with reference to FIGS. 2(a)-2(h). Certain process steps differ from those described in FIGS. 1(a)-1(i) due to the change from silicon to glass wafers. One skilled in the art will appreciate the characteristics of these differing base materials and the processing changes that may be required or tolerated. Initially, in FIG. 2(a), the primary wafer 5, which is glass in this example, is cleaned and marked in accordance with the piraña clean of, for example, H₂SO₄: H₂O₂=4:1, applied for 20 minutes. Next, in FIG. 2(b), chrome/gold or titanium/gold deposition layers are applied. Chrome or titanium 20, is needed as an intermediate layer as discussed above since gold 25 has poor adhesion to glass. The chrome/titanium and gold are sputtered using a standard sputtering machine. Layer details are set forth in Table 7 below. In a preferred embodiment, the chrome/gold combination is used. Alternatively, as suggested above, a chrome/platinum combination may be used.

TABLE 7

Flush the chamber a few times with Argon (Ar)
200 Å Cr or Ti sputter deposition
5000 Å Au sputter deposition

Referring next to FIG. 2(c), a photo resist layer 70 is added by lithography using an appropriate mask. The specifications and recipe are set forth in Table 8 below.

TABLE 8

Spin Shipley ~4 mL S1813 PR
5 s @ 500 rpm, 30 s @ 3000 rpm, ramp 400 rpm/s
Bake 60 s @ 110° C.
Overlay mask
Expose 10 s
Develop 20-40 s in CD-30, (FRESH DEVELOPER)
Bake 30 min @ 120° C.

Next, the electrodes are patterned 75 via etching as shown in FIG. 2(d) pursuant to the specifications and recipe are set forth in Table 9 below.

TABLE 9

Gold etch: Use 80-100 mL of TFS etchant
Immerse 2-5 min on shaker until completely etched
Rinse with DI water, do not dry, proceed immediately to next etch
Chrome etch: mix Transene TFN etchant to DI water
Titanium etch: Transene TFTN etchant @ 90° C.
Immerse 1-2 min until completely etched
Rinse with DI water, dry

Referring to FIG. 2(e), Polymethyl metacrylate (PMMA) 35 is deposited over the patterned electrodes. Table 10 sets forth recipe and formation details.

TABLE 10

Use regular chuck
Cover the entire pattern with 950 PMMA C10
Spin PMMA, use recipe 1: 45 s @ 4500 rpm, ramp 400 rpm/s
Prebake 70 s @ 190° C.

FIGS. 2(f) and 2(g) are snap shots of the wafer during the dicing process, whereby individual sub-wafers 5_{S1} and 5_{S2}, i.e., arrays of monitoring sites, are separated from the larger single wafers. Generally, the glass wafer 5 is attached to the sticky side of tape 80 in order to stabilize during and after dicing. One skilled in the art recognizes that machine and process step variations may be used so long as the wafer is diced so as to yield the individual sub-wafers described herein.

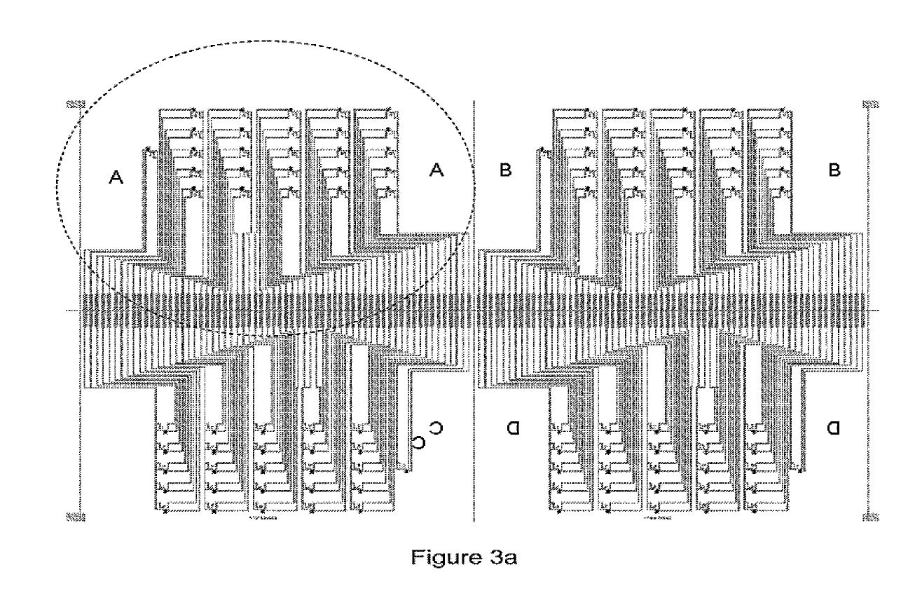

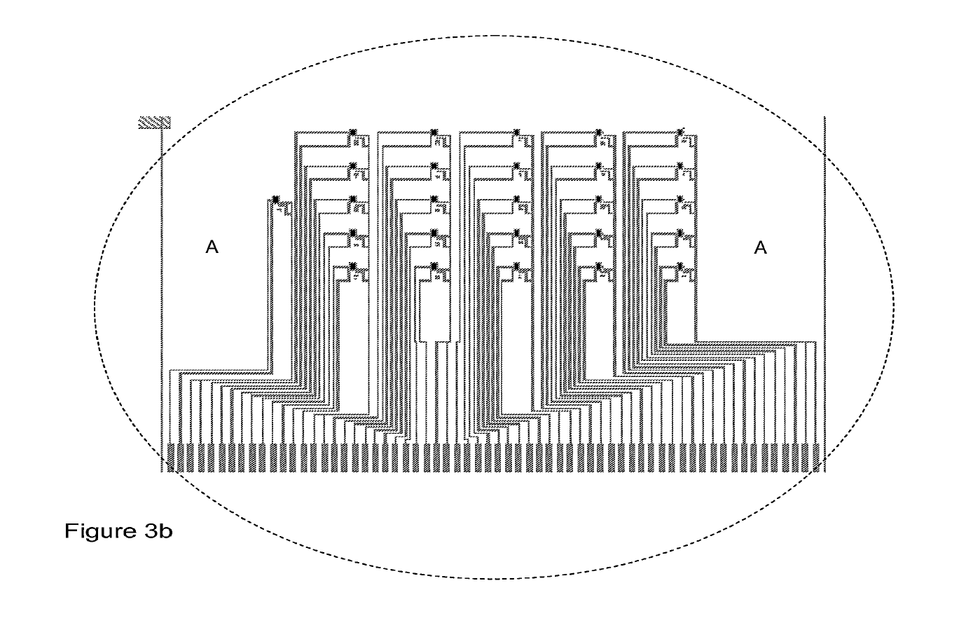

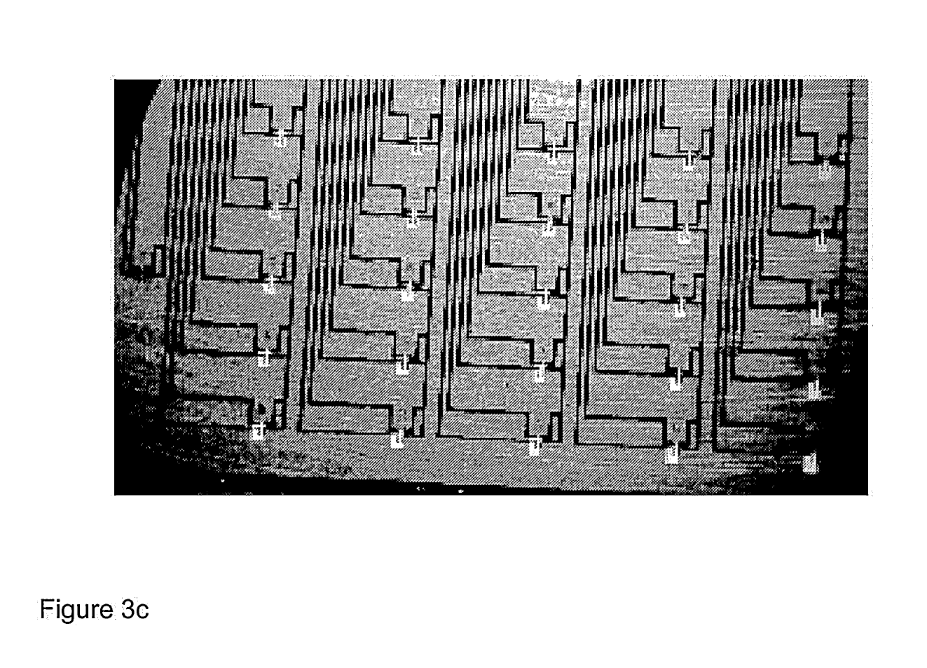

In accordance with FIG. 2(h), reactive ion etching of the PMMA and photoresist layers is employed for each subwafer to expose the underlying electrodes as set forth in Table 11. The referenced shadow mask is shown in FIG. 3c:

TABLE 11

RIE CF₄/O₂ etch, use shadow mask
Etch pads

Finally, glucose oxidase (GOx) is electrochemically deposited through the openings in the PMMA layer. The recipe and steps are identified in Tables 12a and 12b below.

TABLE 12a

To prepare electrolyte solution mix the following in a 10 mL beaker:
9.6 mL phosphate buffer solution (1X)
1 mL 1M KCl solution
78 μL 95% pyrole solution
Use graphite electrode as the counter electrode, Ag/AgNaCl as the
reference electrode and the device as the working electrode
Switch all the heater switches on
Turn the potentiostat on
Choose CV mode and run a scan from −1 V to +1 V @ 200 mV/s

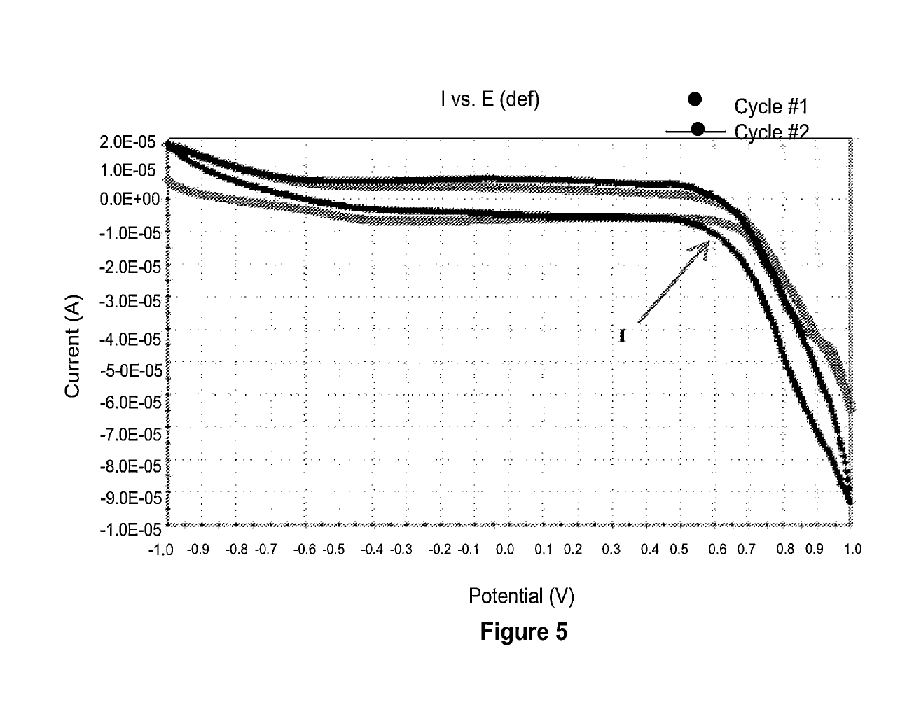

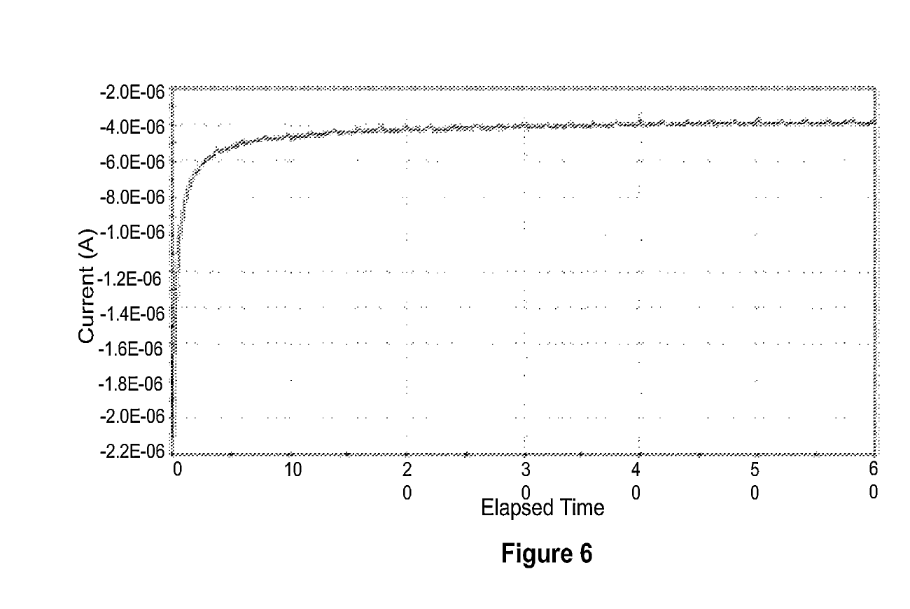

Referring to FIG. 5, the inflection point 1 at approximately 0.6 V shows an increase (inverted scale) in the amount of current that can be passed through the electrode. This technique is used to determine an appropriate voltage for electrodeposition (polarization) to occur. This is the reason there is an increase in the polarization current. This is the voltage at which polypyrole deposition will be performed. Next, in chronoamperometry mode, use a one-step power mode to perform a polypyrole deposition at a voltage selected from the CV curve (0.6 V) for 60 seconds (see FIG. 6).

TABLE 12b
Add 48 μL K₃FeCN₆ + 18 μL GOx
Perform a 10 min one-step chronoamperometric deposition at 0.4 V
Remove the device, rinse with DI water and insert into the connector again
Put it into a beaker with only PBS solution
Run a CV from −1 V to +1 V @ 200 mV/s

Alternatively, the PPy and GOx may be deposited together in a single step of 0.6 volts for 1 minute.

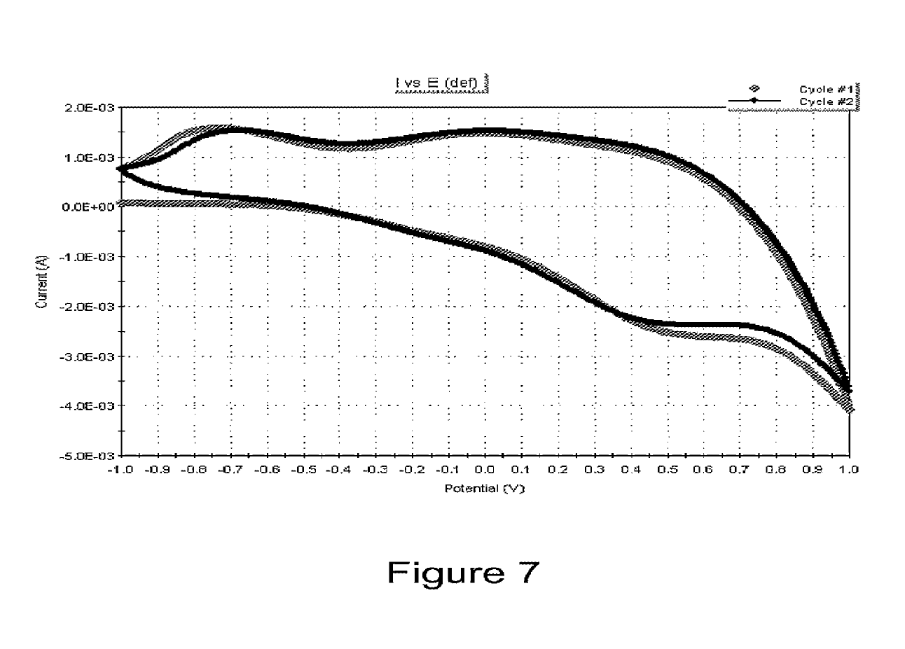

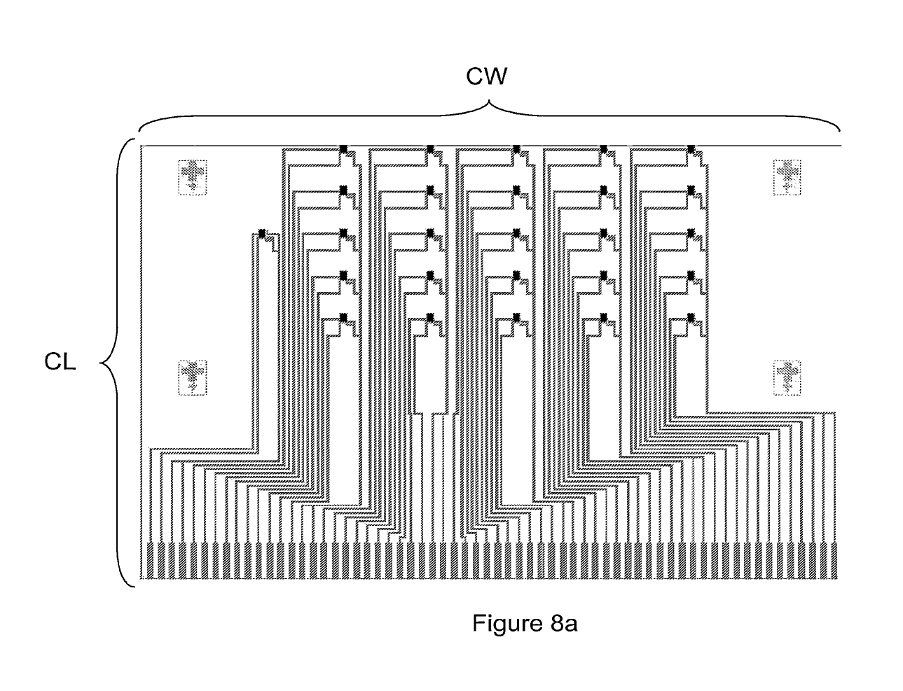

Next, referring to FIG. 7, a CV scan is run from −1 V to +1 V to verify the deposition of polypyrole and also indicate the reduction potential of the PPy GOx matrix. In FIG. 7, the CV was run for two cycles. The voltage 0.4 V is determined to be the voltage at which subsequent testing is performed and is also the polarization potential used in the a polarization step. More specifically, a polarization step is used to eliminate built-in charges between the sensor's metal layer and the conducting PPy matrix. In this step, the potential determined from the last CV scan, i.e., 0.4 V is maintained across the PPy Gox film until a steady current is obtained. This steady state signal is also called the background current and serves as baseline for future measurements.

In an additional step (not illustrated), the second electrode is opened up in the PMMA layer using the same oxygen plasma specifications and mask as described in Table 11.

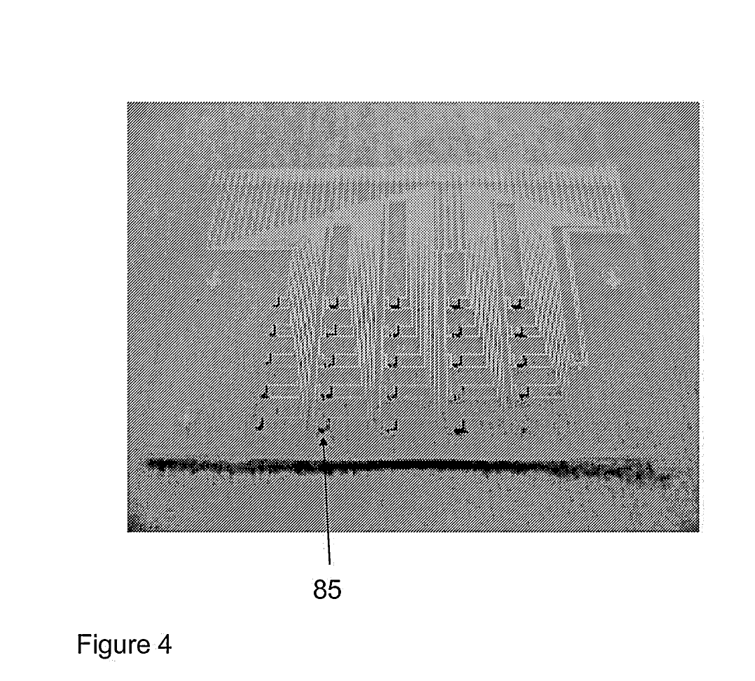

Accordingly, resulting from the process steps described above are multiple transdermal monitoring devices having the architecture shown in FIG. 4. FIG. 4 illustrates an exemplary subwafer 5_{S1} post GOx. Subwafer 5_{S1} as shown includes a five by five array of individual monitoring sites 85. Each individual monitoring site 85 includes an electrically controllable heater for ablating the skin of an individual to access interstitial fluid and a sensing area for electrochemically sensing an amount of an analyte, e.g., glucose, in the interstitial fluid. As will be readily apparent to one skilled in the art of glucose monitoring, such an array would be useful in the daily monitoring routines of individuals suffering with diabetes.

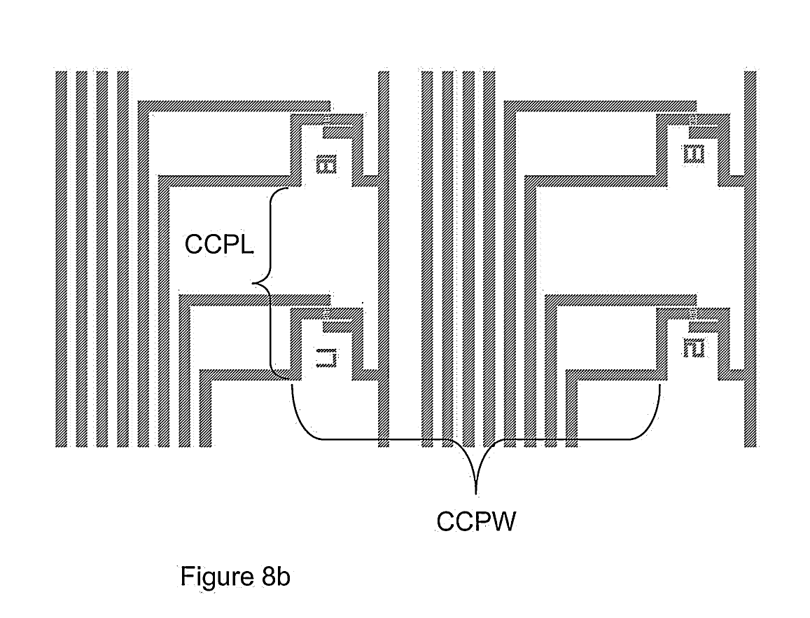

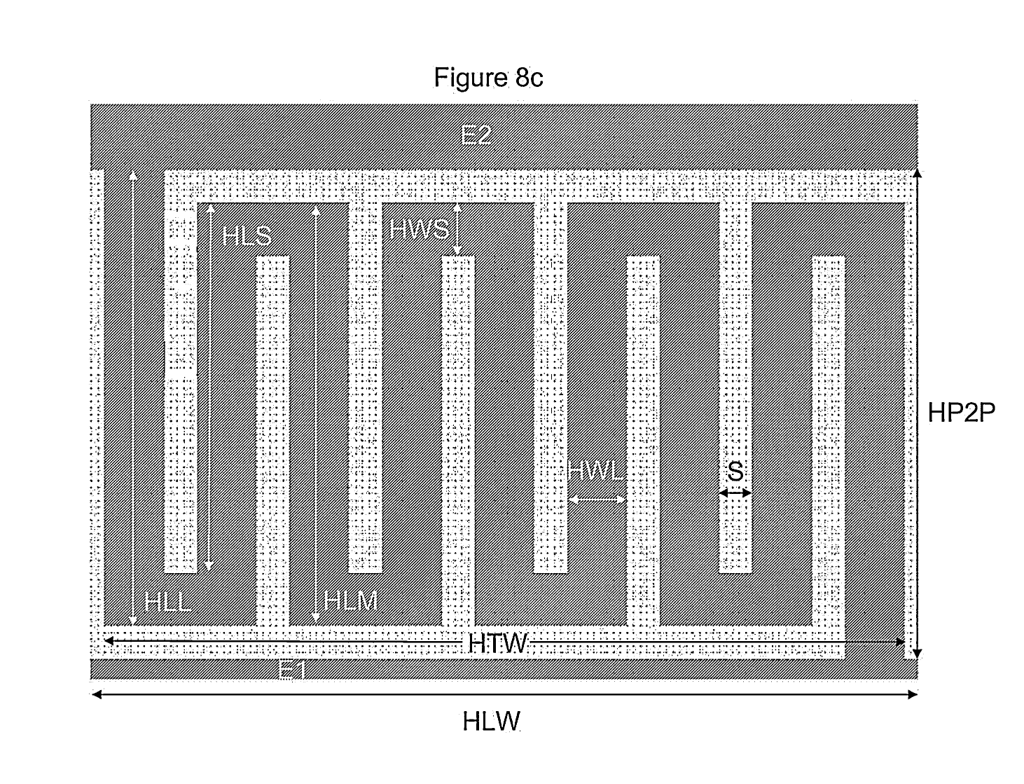

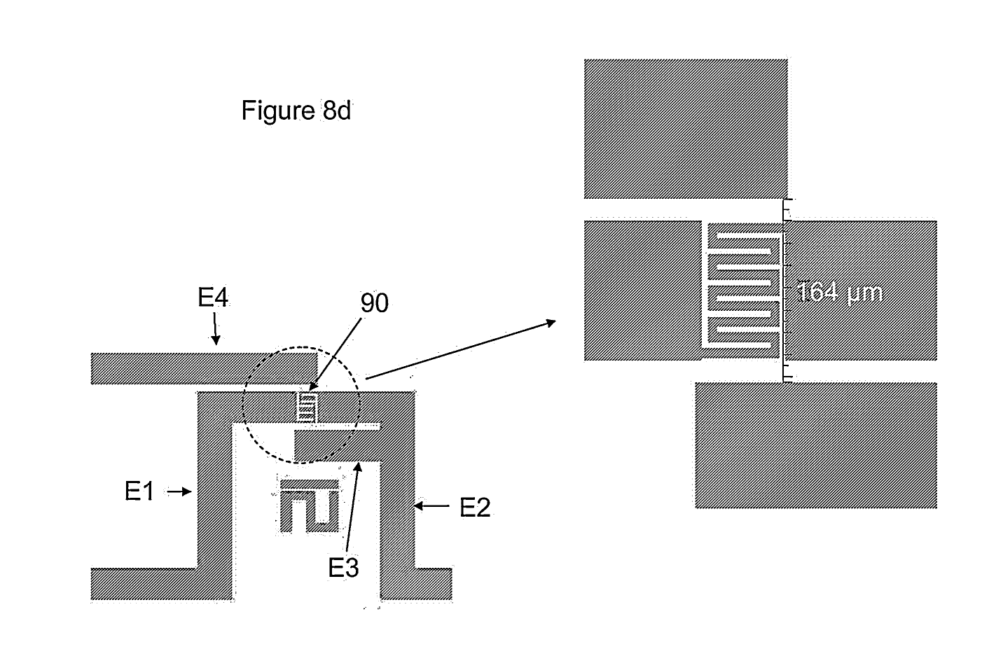

The device dimensions in the examples described here are in the micron range. More specifically, and by way of example, various dimensions of an individual device constructed in accordance with the process in FIGS. 2a through 2h are shown in FIGS. 8a through 8d. Referring to FIG. 8a, chip width (CW) is approximately 32,000 microns and chip length (CL) is approximately 23,000 microns. Referring to FIG. 8b, chip-to-chip pitch width (CCPW) is approximately 4,000 microns and chip length (CCPL) is approximately 2100 microns. Referring to FIG. 8c, serpentine heater dimensions are as follows: the heater lead width (HLW) is approximately 125 microns; the heater pad to pad (HP2P) is approximately 74 microns; the heater total width (HTW) is approximately 121 microns; the space between elements (S) is approximately 5 microns; the short heater width (HWS) is approximately 8 microns; the long heater width (HWL) is approximately 9 microns; the short heater length (HLS) is approximately 48 microns; the medium heater length (HLM) is approximately 64 microns; and the long heater length (HLL) is approximately 69 microns.

FIG. 8d illustrates additional dimensions between various electrodes that are available for use with the processes described herein. More particularly, as shown, E1, E2, E3 and E4 illustrate different portions of electrode material. As discussed further herein, E3 is an extension of E2. Further, in a preferred configuration, E1 and E2 are initially part of a closed-circuit system along with the serpentine conductor, i.e., heater 90. As shown in. FIG. 8c, the distance between E1 and E2 is approximately 74 microns (HP2P). As shown in FIG. 8d, the distance between E3 and E4 is approximately 164 microns.

Accordingly, taking the specific embodiment of FIG. 8a-8d as an exemplary device, the individual monitoring sites (exclusive of electrodes/leads) are at least the size of the heater, i.e., approximately HTVV×HP2P which is 121 microns×74 microns=8954 microns². Generally, an active area of approximately 50×50 microns=2500 microns² is sufficient to ablate the stratum corneum of the subject and access a sufficient amount of interstitial fluid to perform desired glucose monitoring. The depth of the active area is approximately 40 microns. One skilled in the art recognizes that the these dimensions may vary in accordance with manufacturing tolerances and other considerations. The dimension may be optimized in accordance with intended location of the device on the user's body and other attributes of the user, e.g., skin tone, type, follicle structure and the like. This optimization is within the scope of the invention.

In a preferred operation, the process for taking a glucose reading requires only two of the four electrode portions, E1 and E2. In this preferred operation, an approximately 3 volt initial pulse is applied to the heater through electrode portions E1 and E2 which initially forms a closed-circuit configuration. This initial pulse causes the serpentine conductive material forming the heater to heat up and ultimately said heat transfers to the skin of the subject with is in thermal contact therewith. This heat thermally ablates a portion of the stratum corneum, allowing interstitial fluid to come into contact with the device. This initial approximately 3 volt pulse also acts to open or “blow” the heater and open the previously closed circuit, thus forming an open-circuit configuration. This results in the formation of two separate and electrically isolated electrodes. A second voltage pulse of approximately 0.3 to 0.4 volts is applied to the open circuit and measurement of current occurs between E1 and E2, at least one of which has been modified with a sensing material, i.e., GOx and PPY matrix. The sensing layer is in communication with a measurement device, e.g., integrated circuitry including a microprocessor, for receiving the measurement data from the sensing layer. This measurement data may be in the form of current readings and is indicative of an amount of analyte, e.g., glucose, in the interstitial fluid of the user. In this embodiment, electrode portions E3 and E4 are not used.

In an alternative embodiment, the initial 3 volt pulse may not open the circuit. In this case, a second approximately 3 volt pulse may be applied. Once the circuit is opened, the measurement pulse and processes described above are applicable.

In an alternative embodiment, after the approximately 3 volt pulse is applied to the heater through electrode portions E1 and E2 to cause the heater to ablate the stratum corneum and release the interstitial fluid; electrode portions E3 and E4 are used as the measuring electrodes for measuring current resulting from the electrochemical reaction of the analyte with the sensing layer in response to a voltage pulse of approximately 0.3 to 0.4 volts applied thereto. Similarly, if for some reason the circuit simply does not open, electrode portions E3 and E4 may be used as the measuring electrodes for measuring current resulting from the electrochemical reaction of the analyte with the sensing layer in response to a voltage pulse of approximately 0.3 to 0.4 volts applied thereto.

Integrated circuitry (IC), including radio frequency (RF) communication capability, may be included as part of the individual device in order to transmit data readings to a remote location. By way of example, this transmission may be facilitated as part of a home area network (HAN) in a first instance, e.g., using protocols such as those described as part of the Zigbee standards. Further still, the data readings may be further transmitted outside of the HAN in accordance with a home health or telehealth communications system using existing wide area networks (WANs) such as the Internet.

The present embodiments provide for other advantages over the existing art in addition to the non-invasive features. For example, the present device does not require a separate reservoir for collecting interstitial fluid, an additional perfusion liquid to mix with the interstitial fluid or any additional means for affirmatively suctioning or pulling in the interstitial fluid. The device is structured such that the natural dispersion of the interstitial fluid from the heated area is sufficient to trigger an electrochemical response with the GOx.

The heaters can be formulated for a single use, wherein, once heated, the heating material is essentially blown or destroyed for that particular individual site. Alternatively, the heaters could be structured for multiple uses, which require smaller voltage pulses to reach the desired temperature to ablate the stratum corneum and release the interstitial fluid.

One skilled in the art recognizes the other areas of application for the devices described herein. While the examples specifically described herein are directed to glucose monitoring, adaptations could be made to ascertain other information from the bio-molecules and bio-markers in the interstitial fluid. For example, the individual sites could monitor for infectious disease (microbial, fungal, viral); hazardous compounds; heart or stroke indicators (troponin, C-reactive protein); chemical or biological toxins; cancer markers (PSA, estrogen); drug efficacy and dosing (metabolites): and the like. Such applications of the device as described are considered to be within the scope of the present invention.

1. A device containing at least two individually controllable sites for electrochemically monitoring an analyte in interstitial fluid of a user comprising:
a glass substrate having formed thereon at each of the at least two individually controllable sites:
a serpentine conductive, pattern attached at a first and second ends thereof to electrode material in a closed-circuit configuration for receiving a first predetermined voltage applied thereto in order to;
i. thermally ablate a stratum corneum of a user's skin to access the interstitial fluid of the user and
ii. form an open-circuit configuration including first and second portions of the electrode material that are electrically isolated from each other;
a sensing area deposited on at least one of the first and second portions of the electrode material; and
a measuring component for receiving individual measurement data from the sensing area in response to a second predetermined voltage applied to the open circuit configuration of each of the at least two individually controlled sites in the open-circuit configuration, wherein the individual measurement data is indicative of an amount of the analyte in the interstitial fluid of the user.

2. The device according to claim 1, wherein the sensing area includes a matrix of polypyrole (PPY) and glucose oxidase (GOx).

3. The device according to claim 2, wherein an amount of polypyrole in the matrix is in accordance with one-step chronoamperometric deposition at 0.6 volts for 60 seconds.

4. The device according to claim 2, wherein an amount of glucose oxidase in the matrix is in accordance with one-step chronoamperometric deposition at 0.4 volts for 10 minutes.

5. The device according to claim 1, wherein the first predetermined voltage is approximately 3 volts.

6. The device according to claim 1, wherein the stratum corneum is ablated to a depth of approximately 40 microns.

7. The device according to claim 1, wherein the electrode material comprises an adhesion layer deposited on the glass substrate and a conductive layer deposited on the adhesion layer.

8. The device according to claim 7, wherein the adhesion layer is comprised of at least one of titanium and chrome.

9. The device according to claim 7, wherein the conductive layer is comprised of at least one of gold and platinum.

10. The device according to claim 1, wherein the analyte is glucose.

11. The device according to claim 1, wherein a distance between the first and second portions of the electrode material is equal to or less than 74 microns.

12. The device according to claim 1, wherein an area of the serpentine conductive pattern is equal to or less than 8954 microns².

13. A process for electrochemically monitoring an analyte in interstitial fluid of a user comprising:
applying a first predetermined voltage to a closed-circuit device located proximate to a portion of skin of the user that includes a serpentine conductive pattern attached at a first and second ends thereof to electrode material in order to:
i. thermally ablate a stratum corneum of a user's skin to access the interstitial fluid of the user; and
ii. separate the electrode material to form an open-circuit device including first and second portions of the electrode material that are electrically isolated from each other;
applying a second predetermined voltage to the open-circuit device which is electrically contacted with the interstitial fluid; and
receiving at a measuring component from a sensing area located on at least one of the first and second portions of the electrode material, measurement data indicative of an amount of the analyte in the interstitial fluid of the user.

14. The process according to claim 13, wherein the first predetermined voltage is approximately 3.0 volts.

15. The process according to claim 13, wherein the second predetermined voltage is approximately 0.3-0.4 volts.

16. A device containing at least two individually controllable sites for electrochemically monitoring an analyte in interstitial fluid of a user comprising:
a glass substrate having formed thereon at each of the at least two individually controllable sites:
a serpentine conductive pattern attached at a first and second ends thereof to electrode material in a closed-circuit configuration for receiving a predetermined voltage applied thereto in order to thermally ablate a stratum corneum of a user's skin to access the interstitial fluid of the user;
a sensing area located on at least a portion of the electrode material; and
first and second measuring electrodes for obtaining measurement data from the sensing area;
a measuring component for receiving individual measurement data from:the first and second measuring electrodes of each of the at least two individually controlled sites, wherein the individual measurement data is indicative of an amount of the analyte in the interstitial fluid of the user.

17. The device according to claim 16, wherein the sensing area includes a matrix of polypyrole (PPY) and glucose oxidase (GOx).

18. The device according to claim 17, wherein an amount of polypyrole in the matrix is in accordance with one-step chronoamperometric deposition at 0.6 volts for 60 seconds.

19. The device according to claim 17, wherein an amount of glucose oxidase in the matrix is in accordance with one-step chronoamperometric deposition at 0.4 volts for 10 minutes.

20. The device according to claim 16, wherein the first predetermined voltage is approximately 3 volts.

21. The device according to claim 16, wherein the stratum corneum is ablated to a depth of approximately 40 microns.

22. The device according to claim 16, wherein the electrode material and the first and second measuring electrodes comprises an adhesion layer deposited on the glass substrate and a conductive layer deposited on the adhesion layer.

23. The device according to claim 19, wherein the adhesion layer is comprised of at least one of titanium and chrome.

24. The device according to claim 19, wherein the conductive layer is comprised of at least one of gold and platinum.

25. The device according to claim 16, wherein the analyte is glucose.

26. The device according to claim 16, wherein a distance between the first and second measuring electrodes is equal to or less than 164 microns.

27. The device according to claim 16, wherein an area of the serpentine conductive pattern is equal to or less than 8954 microns².

28. A process for electrochemically monitoring an analyte in interstitial fluid of a user comprising:
applying a first predetermined voltage to a closed-circuit device located proximate to a portion of skin of the user that includes a serpentine conductive pattern attached at a first and second ends thereof to electrode material in order to thermally ablate a stratum corneum of a user's skin to access the interstitial fluid of the user and form an open-circuit device;
applying a second predetermined voltage to the open-circuit device which is in electrical contact with the interstitial fluid;
measuring an electrochemical response resulting from an interaction of the analyte with a sensing layer on a portion of the electrode material; and,
receiving at a measuring component from the open circuit device, measurement data indicative of an amount of the analyte in the interstitial fluid of the user.

29. The process according to claim 28, wherein the first predetermined voltage is approximately 3.0 volts.

30. The process according to claim 28, wherein the second predetermined voltage is approximately 0.3-0.4 volts.

31. The process according to claim 28, wherein if an initial application of the first predetermined voltage to a closed-circuit device docs not form the open-circuit device, the first predetermined voltage is applied a second time.

32. A process for forming a device containing at least two individually controllable sites for electrochemically monitoring glucose in interstitial fluid of a user comprising:
depositing a first layer of one of chrome or titanium on a glass substrate;
depositing a second layer of one of gold or platinum on the first layer of chrome;
patterning the first and second layers in a first predetermined pattern to form multiple electrodes;
depositing polymethyl methacrylate (PMMA) on the first predetermined pattern;
patterning the PMMA in a second predetermined pattern, wherein at least a portion of the first predetermined pattern is exposed; and
electrochemically depositing polypyrole (PPY) and glucose oxidase (GOx) on the exposed portion of the first predetermined pattern in a single step.

33. The process according to claim 32, further comprising further patterning remaining PMMA in third predetermined pattern to expose at least one of the multiple electrodes.
